SEQUENCE LISTING (17 sequences)

SEQ ID NO: 1 - RNA, 42 nt, synthetic construct
  source 1..42: mol_type = other RNA
gacttcgccctgcccgtggagaagcccctgtgcctgcagaga

SEQ ID NO: 2 - protein, 14 aa, synthetic construct
DFALPVEKPLCLQR

SEQ ID NO: 3 - RNA, 42 nt, synthetic construct
  source 1..42: mol_type = other RNA
accggcgacttcgccctgcccgtggccagtctggcctgcaga

SEQ ID NO: 4 - protein, 14 aa, synthetic construct
TGDFALPVASLACR

SEQ ID NO: 5 - RNA, 42 nt, synthetic construct
  source 1..42: mol_type = other RNA
gacttcgctcttccagttgagaagcccttgtgcctgcagaga

SEQ ID NO: 6 - protein, 14 aa, synthetic construct
DFALPVEKPLCLQR

SEQ ID NO: 7 - RNA, 42 nt, synthetic construct
  source 1..42: mol_type = other RNA
actggcgacttcgctcttccagttgcctcacttgcctgccga

SEQ ID NO: 8 - protein, 14 aa, synthetic construct
TGDFALPVASLACR

SEQ ID NO: 9 - RNA, 42 nt, synthetic construct; identical to SEQ ID NO: 1
  source 1..42: mol_type = other RNA

SEQ ID NO: 10 - protein, 14 aa, synthetic construct
DFALPVEKPLCLQR

SEQ ID NO: 11 - RNA, 42 nt, synthetic construct; identical to SEQ ID NO: 3
  source 1..42: mol_type = other RNA

SEQ ID NO: 12 - protein, 14 aa, synthetic construct
TGDFALPVASLACR

SEQ ID NO: 13 - RNA, 327 nt, synthetic construct
  source 1..327: mol_type = other RNA
gccagccccctgatgggggcgacactccaccatgaatcactcccccggcgggtctacgtcttcacgcagaaagcgcctagccatggcgctagtatgagtgtcgtgcagccgccgggaccccccctcccgggagagccatagcggtctgcggaaccggtgcaccggaatgccaggacgaccgggtcctttcttggatcaacccgctcaatgcctggagatttgggcgtgcccccgcagaccgcaagccgtagtgttgggtcgcgaaggccttgtggtactgcctgatagggtgcttgcgagtgcccctttaggtctcgtagaccgtgc

SEQ ID NO: 14 - RNA, 131 nt, synthetic construct
  source 1..131: mol_type = other RNA
gctcgctttcttgctgtccaatttctattaaaggtcctttgttccctaagtccaactactaaactgagggatattacaaagggccttgagcatctggattctgcctaataaaaaacatttattttcattgc

SEQ ID NO: 15 - RNA, 506 nt, synthetic construct
  source 1..506: mol_type = other RNA
gccagccccctgatgggggcgacactccaccatgaatcactcccccggcgggtctacgtcttcacgcagaaagcgcctagccatggcgctagtatgagtgtcgtgcagccgccgggaccccccctcccgggagagccatagcggtctgcggaaccggtgcaccggaatgccaggacgaccgggtcctttcttggatcaacccgctcaatgcctggagatttgggcgtgcccccgcagaccgcaagccgtagtgttgggtcgcgaaggccttgtggtactgcctgatagggtgcttgcgagtgcccctttaggtctcgtagaccgtgcatggacttcgccctgcccgtggagaagcccctgtgcctgcagagataagctcgctttcttgctgtccaatttctattaaaggtcctttgttccctaagtccaactactaaactgagggatattacaaagggccttgagcatctggattctgcctaataaaaaacatttattttcattgc

SEQ ID NO: 16 - RNA, 506 nt, synthetic construct
  source 1..506: mol_type = other RNA
gccagccccctgatgggggcgacactccaccatgaatcactcccccggcgggtctacgtcttcacgcagaaagcgcctagccatggcgctagtatgagtgtcgtgcagccgccgggaccccccctcccgggagagccatagcggtctgcggaaccggtgcaccggaatgccaggacgaccgggtcctttcttggatcaacccgctcaatgcctggagatttgggcgtgcccccgcagaccgcaagccgtagtgttgggtcgcgaaggccttgtggtactgcctgatagggtgcttgcgagtgcccctttaggtctcgtagaccgtgcatgaccggcgacttcgccctgcccgtggccagtctggcctgcagataagctcgctttcttgctgtccaatttctattaaaggtcctttgttccctaagtccaactactaaactgagggatattacaaagggccttgagcatctggattctgcctaataaaaaacatttattttcattgc

SEQ ID NO: 17 - protein, 7 aa, synthetic construct
RRRRRRG

(19) United States
(12) Patent Application Publication
(10) Pub. No.: US 2024/0050542 A1
(43) Pub. Date: Feb. 15, 2024

(54) MRNA THERAPEUTIC VACCINE FOR TREATMENT OF ATHEROTHROMBOSIS

(71) Applicant: ATHEROVAX. LLC, Lebanon, OR (US)

(72) Inventors: Patrick IVERSEN, Grand Junction, CO (US); Nodar KIPSHIDZE, New York, NY (US); Nickolas KIPSHIDZE, New York, NY (US)

(21) Appl. No.: 18/332,873

(22) Filed: Jun. 12, 2023

Related U.S. Application Data
(60) Provisional application No. 63/351,648, filed on Jun. 13, 2022.

(51) Int. Cl. A61K 39/00

(52) U.S. Cl. CPC A61K 39/0008; A61K 2039/53; A61K 2039/55555; A61K 2039/545; A61K 2039/54

(57) ABSTRACT

Described herein are mRNA-based and peptide-based therapeutic vaccines comprising modified TNFR2 sequences complementary to variants of Homo sapiens TNFR2 genes and methods for treating subjects having atherosclerosis.

### CROSS-REFERENCE TO RELATED APPLICATIONS

This application claims priority to U.S. Provisional Patent Application No. 63/351,648 filed on Jun. 13, 2022, which is incorporated by reference herein in its entirety.

### REFERENCE TO SEQUENCE LISTING

This application was filed with a Sequence Listing XML in ST.26 XML format accordance with 37 C.F.R. § 1.831. The Sequence Listing XML file submitted in the USPTO Patent Center, “054228-9017-US02_sequence_listing_xml_30-MAY-2023.xml,” was created on May 30, 2023, contains 17 sequences, has a file size of 17.1 Kbytes, and is incorporated by reference in its entirety into the specification.

### TECHNICAL FIELD

Described herein are mRNA-based and peptide-based therapeutic vaccines comprising modified TNFR2 sequences complementary to variants of Homo sapiens TNFR2 genes and methods for treating subjects having atherosclerosis.

BACKGROUND

Atherosclerosis is a systemic dysfunctional endothelial, focal occurring, chronic inflammatory, fibro-proliferative, prothrombotic, angiogenic, multifactorial disease of the arterial intima caused by the retention of modified low-density lipoproteins, hemodynamic, and reductive-oxidative (redox) stress. There is no question that atherosclerosis is a systemic dysfunctional endothelial disease. Inflammation in the setting of the vulnerable plaque is the body's natural protective response to ischemic injury of the vessel wall providing oxygen and metabolic nourishment as the intima undergoes a positive outward remodeling and thickening, while at the same time may contribute to plaque growth through the response to injury mechanism to intraplaque hemorrhage (IPH). As the numbers of these “malignant like” microvessels increase within the plaque, the numbers of IPH increase as a result and contribute to the instability of the atherosclerotic plaque. Even though the IPH may be clinically silent, it may result in progression of atherosclerosis.

TNFα is a small 233 amino acid protein released by macrophages resulting in inflammatory responses. TNFα can be membrane-bound (mTNFα) or as a soluble form (sTNFα). sTNFα is the product of proteolytic cleavage of the mTNFα by a metalloprotease TNF converting enzyme (TACE, also called ADAM17). The sTNFα is associated as a homotrimeric cytokine. The TNFα binds two receptors, TNFR1 and TNFR2. TNFR1 is expressed on most cell types and signaling leads to pro-inflammatory and apoptosis responses. TNFR2 is expressed primarily on endothelial, epithelial, and some immune cells, and signaling is anti-inflammatory and promotes cell proliferation. Dysregulation of TNFα is associated with Alzheimer's disease, cancer, depression, psoriasis, and inflammatory bowel disease. TNFα is used as an immunostimulant drug, tasonermin, to treat various inflammatory diseases. TNFα inhibitors include monoclonal antibodies such as Remicade (infliximab), Humera (adalimumab), Cimzia (certolizumab), or as a receptor fusion decoy as Enbrel (etanercept).

In contrast, soluble TNF receptor II (sTNFR2) is associated with increased mortality and morbidity in many human diseases but there are no therapeutic interventions targeting sTNFR2. It is not clear that the role of the sTNFR2 in chronic inflammatory disease is to prolong the actions of TNFα compared to sequestration of TNFα in acute inflammation. The soluble TNFR2 is synthesized as a splice variant of TNFR2. The membrane-binding domain related to exon 7 (a cassette exon) is excluded from the mature mRNA resulting in a truncated protein with no membrane-binding domain. A soluble TNFR2 can be created by peptidase cleavage of amino acids associated with exons 7 and 8, leading to a truncated protein with no membrane-binding domain. In ST-elevation myocardial infarction (STEMI) patients, circulating levels of sTNFR1 and sTNFR2 are associated with infarct size and LV dysfunction. Nilsson et al., PLoS One 8(2): e55477 (2013). sTNFR1 (HR 1.51) and sTNFR2 (HR 1.13) are independently associated with all-cause mortality or an increased risk for cardiovascular events in advanced CKD irrespective of the cause of kidney disease. Neirynck et al., PLoS One 10(3): e0122073 (2015). Increased concentrations of circulating sTNFR1 and sTNFR2 were associated with increased risks of cardiovascular events and mortality in patients with stable coronary heart disease. Carlsson et al., J. Am. Heart Assoc. 7(9) :e008299 (2018). A link between sTNFR2 levels and altered epigenetic methylation and gene expression involves differential methylation of 168 CpGs, many involved in antigen processing and presentation. Mendelson et al., Front. Pharmacol. 9: 207 (2018).

There is, therefore, a need for a method to locally regress or stabilize plaque in the main blood supply. There is a further need for a treatment modality of vulnerable and stable atherosclerotic plaques, including the vasa vasorum of the atherosclerotic plaque. What is needed is a therapeutic vaccine for systemic or local administration to an atherosclerotic plaque to treat or prevent atherosclerosis.

### SUMMARY

One embodiment described herein is an RNA vaccine comprising a nucleotide sequence selected from one or more of SEQ ID NO: 5, 7, 9, 11, or 13-16 and one or more pharmaceutically acceptable excipients. In one aspect, the nucleotide sequence is selected from SEQ ID NO: 15 or 16. In another aspect, the nucleotide sequence comprises one or more modifications to the ribose sugar, the nucleotide base, the 5′-terminus, the 3′-terminus, or combinations thereof.

Another embodiment described herein is a peptide vaccine comprising a polypeptide sequence selected from one or more of SEQ ID NO: 6, 8, 10, or 12 and one or more pharmaceutically acceptable excipients. In one aspect, the polypeptide sequence is SEQ ID NO: 6. In another aspect, the polypeptide sequence comprises one or more modifications to the peptide backbone, the amino acid side chains, the N-terminus, the C-terminus, or combinations thereof.

Another embodiment described herein is an RNA vaccine or peptide vaccine as described herein in a nanoparticle formulation. In one aspect, the vaccine is immediately released or controlled released.

Another embodiment described herein is a vascular stent, comprising the RNA vaccine or peptide vaccine as described herein. In one aspect, the vaccine is immediately released or controlled released.

Another embodiment described herein is leaflets of a heart valve, comprising the RNA vaccine or peptide vaccine as described herein. In one aspect, the vaccine is immediately released or controlled released.

Another embodiment described herein is a drug eluting balloon, comprising the, comprising the RNA vaccine or peptide vaccine as described herein. In one aspect, the vaccine is immediately released or controlled released.

Another embodiment described herein is a method for treating atherothrombosis, the method comprising, administering a therapeutically effective amount of comprising the, comprising the RNA vaccine or peptide vaccine as described herein, implanting the vascular stent as described herein, the heart valve as described herein, the drug eluting balloon as described herein to a subject in need thereof. In one aspect, the vaccine is immediately released or controlled released. In another aspect, the vaccine is administered four times over a period of 16 weeks. In another aspect, the vaccine is administered at the initial administration, at 4 weeks, at 10 weeks, and at 16 weeks. In another aspect, the vaccine dose is 30 μg to 100 μg mg.

Another embodiment described herein is a method for treating atherothrombosis, the method comprising, administering a therapeutically effective amount of a therapeutically effective amount of comprising the, comprising the RNA vaccine or peptide vaccine as described herein locally directly to atherosclerotic plagues using micro catheters. In one aspect, the vaccine is immediately released or controlled released. In another aspect, the vaccine is administered four times over a period of 16 weeks. In another aspect, the vaccine is administered at the initial administration, at 4 weeks, at 10 weeks, and at 16 weeks. In another aspect, the vaccine dose is 30 μg to 100 μg mg.

### DESCRIPTION OF THE DRAWINGS

FIG. 1 shows exon reading frames for TNFR2, Δ7TNFR2, and Δ7,8TNFR2.

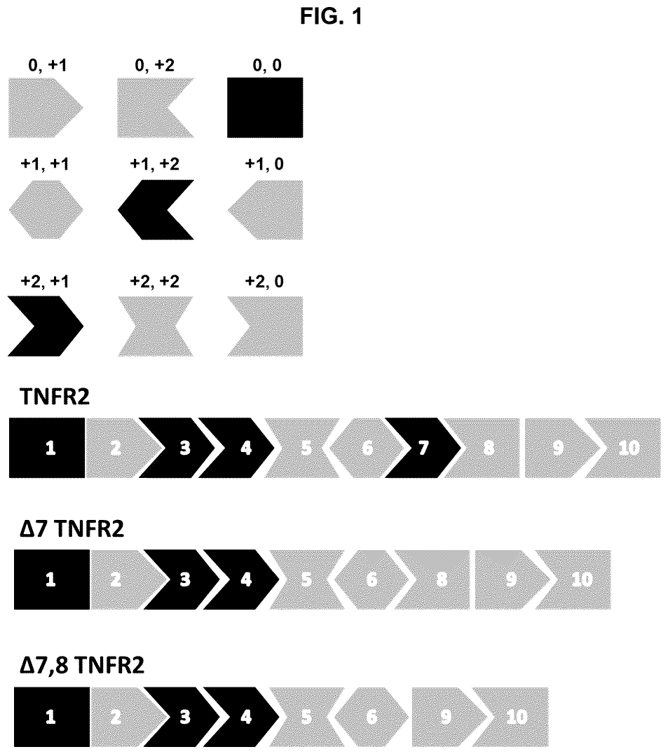

FIG. 2 show sites of codon optimization for Δ7TNFR2 and Δ7,8TNFR2.

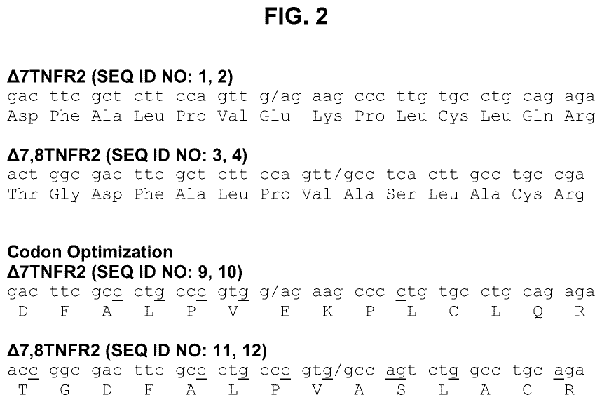

### DETAILED DESCRIPTION

Unless otherwise defined, all technical and scientific terms used herein have the same meaning as commonly understood by one of ordinary skill in the art. For example, any nomenclatures used in connection with, and techniques of, cell and tissue culture, molecular biology, immunology, microbiology, genetics, and protein and nucleic acid chemistry and hybridization described herein are well known and commonly used in the art. In case of conflict, the present disclosure, including definitions, will control. Exemplary methods and materials are described below, although methods and materials similar or equivalent to those described herein can be used in practice or testing of the embodiments and aspects described herein.

As used herein, the terms “amino acid,” “nucleotide,” “polynucleotide,” “vector,” “polypeptide,” and “protein” have their common meanings as would be understood by a biochemist of ordinary skill in the art. Standard single-letter nucleotides (A, C, G, T, U) and standard single letter amino acids (A, C, D, E, F, G, H, I, K, L, M, N, P, Q, R, S, T, V, W, or Y) are used herein.

As used herein, the terms such as “include,” “including,” “contain,” “containing,” “having,” and the like mean “comprising.” The present disclosure also contemplates other embodiments “comprising,” “consisting of,” and “consisting essentially of,” the embodiments or elements presented herein, whether explicitly set forth or not.

As used herein, the term “a,” “an,” “the,” and similar terms used in the context of the disclosure (especially in the context of the claims) are to be construed to cover both the singular and plural unless otherwise indicated herein or clearly contradicted by the context. In addition, “a,” “an,” or “the” means “one or more” unless otherwise specified.

As used herein, the term “or” can be conjunctive or disjunctive.

As used herein, the term “substantially” means to a great or significant extent, but not completely.

As used herein, the term “about” or “approximately” as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In one aspect, the term “about” refers to any values, including both integers and fractional components that are within a variation of up to ±10% of the value modified by the term “about.” Alternatively, “about” can mean within 3 or more standard deviations, per the practice in the art. Alternatively, such as with respect to biological systems or processes, the term “about” can mean within an order of magnitude, in some embodiments within 5-fold, and in some embodiments within 2-fold, of a value. As used herein, the symbol “-” means “about” or “approximately.”

All ranges disclosed herein include both end points as discrete values as well as all integers and fractions specified within the range. For example, a range of 0.1-2.0 includes 0.1, 0.2, 0.3, 0.4 . . . 2.0. If the end points are modified by the term “about,” the range specified is expanded by a variation of up to ±10% of any value within the range or within 3 or more standard deviations, including the end points.

As used herein, the terms “active ingredient” or “active pharmaceutical ingredient” refer to a pharmaceutical agent, active ingredient, compound, or substance, compositions, or mixtures thereof, that provide a pharmacological, often beneficial, effect.

As used herein, the terms “control,” or “reference” are used herein interchangeably. A “reference” or “control” level may be a predetermined value or range, which is employed as a baseline or benchmark against which to assess a measured result. “Control” also refers to control experiments or control cells.

As used herein, the term “dose” denotes any form of an active ingredient formulation or composition, including cells, that contains an amount sufficient to initiate or produce a therapeutic effect with at least one or more administrations. “Formulation” and “composition” are used interchangeably herein.

As used herein, the term “prophylaxis” refers to preventing or reducing the progression of a disorder, either to a statistically significant degree or to a degree detectable by a person of ordinary skill in the art.

As used herein, the terms “effective amount” or “therapeutically effective amount,” refers to a substantially non-toxic, but sufficient amount of an agent, composition, or cell(s) being administered to a subject that will prevent, treat, or ameliorate to some extent one or more of the symptoms of the disease or condition being experienced or that the subject is susceptible to contracting. The result can be the reduction or alleviation of the signs, symptoms, or causes of a disease, or any other desired alteration of a biological system. An effective amount may be based on factors individual to each subject, including, but not limited to, the subject's age, size, type or extent of disease, stage of the disease, route of administration, the type or extent of supplemental therapy used, ongoing disease process, and type of treatment desired.

As used herein, the term “subject” refers to an animal. Typically, the subject is a mammal. A subject also refers to primates (e.g., humans, male or female; infant, adolescent, or adult), non-human primates, rats, mice, rabbits, pigs, cows, sheep, goats, horses, dogs, cats, fish, birds, and the like. In one embodiment, the subject is a primate. In one embodiment, the subject is a human.

As used herein, a subject is “in need of treatment” if such subject would benefit biologically, medically, or in quality of life from such treatment. A subject in need of treatment does not necessarily present symptoms, particular in the case of preventative or prophylaxis treatments.

As used herein, the terms “inhibit,” “inhibition,” or “inhibiting” refer to the reduction or suppression of a given biological process, condition, symptom, disorder, or disease, or a significant decrease in the baseline activity of a biological activity or process.

As used herein, “treatment” or “treating” refers to prophylaxis of, preventing, suppressing, repressing, reversing, alleviating, ameliorating, or inhibiting the progress of biological process including a disorder or disease, or completely eliminating a disease. A treatment may be either performed in an acute or chronic way. The term “treatment” also refers to reducing the severity of a disease or symptoms associated with such disease prior to affliction with the disease. “Repressing” or “ameliorating” a disease, disorder, or the symptoms thereof involves administering a cell, composition, or compound described herein to a subject after clinical appearance of such disease, disorder, or its symptoms. “Prophylaxis of” or “preventing” a disease, disorder, or the symptoms thereof involves administering a cell, composition, or compound described herein to a subject prior to onset of the disease, disorder, or the symptoms thereof. “Suppressing” a disease or disorder involves administering a cell, composition, or compound described herein to a subject after induction of the disease or disorder thereof but before its clinical appearance or symptoms thereof have manifest.

The terms below and used herein, have the following meanings, unless otherwise indicated:

“Antigen” is a molecule that will trigger an immune response, abbreviated by “Ag.” An Ag may originate from within the body (a self-protein) or from an external site (non-self). The immune system may not react to self-proteins due to negative selection of T-cells in the thymus during development.

“Negative selection” is a process in which lymphocytes, capable of strong binding with self-protein defined by the major histocompatibility complex (MHC) are removed by receiving an apoptosis signal leading to cell death. Some lymphocytes are phagocytosed by dendritic cells which allows presentation of self-antigens to MHC class II, a requirement for CD4+ T-cell negative selection. Some of these T-cells responding to self-proteins become Treg (T-regulatory) cells. The process is a component of central tolerance prevents formation of cells capable of inducing autoimmune diseases.

An “antibody” is a “Y” shaped protein, immunoglobulin (Ig), with an antigen binding site and an Fc region. Antibodies from humans include several classes or isotypes; IgA, IgD, IgE, IgG, or IgM. The IgG is composed of four polypeptide chains; two heavy chains and two light chains connected by disulfide bonds. The light chains contain one variable domain (VL) and one constant domain (CL), and the heavy chains contain one variable domaine (VH) and three to four constant domains (CH1, CH2, CH3). Structurally, an antibody has two antigen binding fragments (Fab) composed of VL, VH, CL, and CH1 and a Fc fragment forming the trunk of the Y.

A “T-Cell” is a type of white blood cells, a lymphocyte, which plays a central role in the Adaptive immune response. T-cells a differentiated from other lymphocytes by the presence of a T-cell receptor (TCR) on the cell surface. Multiple classes of T-cells are defined; CD8 killer T-cells, CD4 helper T-cells, and regulatory T-cells. Each class of T-cell performs a different function often involving release of cytokines. All T-cells originate from c-kit+Sca1+ hematopoietic stem cells (HSC) that reside in the bone marrow.

An “Epitope” is a structural feature of an antigen that is an antigenic determinant that matches an antibody recognition site.

“Vaccination” is the physical administration of a vaccine

“Immunization” is the provision of immunity by any means, active or passive

“Active immunization” is the administration of agents for induction of immunity that is long-lasting or at times, life-long

“Passive immunization” is the administration of exogenously produced immune substances (e.g., convalescent serum, adoptive transfer of T-cells, or monoclonal antibodies) that dissipates with the turnover of the administered substances

A “vaccine” is the conveyance of antigens to elicit immune responses that are generally protective. Multiple approaches to vaccine design are known including; attenuated virus, inactive virus, protein subunit, DNA vaccines, vectored vaccines, and mRNA vaccines.

“Vaccine adjuvant” is a substance that increases and/or modulates the immune response to a vaccine antigen. Adjuvants can be inorganic compounds (potassium alum, aluminum hydroxide, aluminum phosphate, calcium phosphate hydroxide), oils (paraffin oil, peanut oil, squalene), bacterial products (mycobacterium Boris, toxoids, lipopolysaccharides), plant products (saponins), cytokines (IL-1, IL-2, IL-12), and stimulators of innate immune responses by binding Toll receptors (TLR ligand including CpG motifs).

“RNA” is a ribonucleic acid and a polymeric molecule essential for coding, decoding, regulation, and expression of genes. Cellular organisms use messenger RNA (mRNA) to convert genetic information as guanine (G), uracil (U), adenine (A), and cytosine (C) as triplets into selection of amino acids in synthesis of specific proteins

“RNA vaccine” is a type of vaccine that uses a copy of a messenger RNA (mRNA) to express an antigen to produce an immune response as well as stimulate innate immune responses.

“Open reading frame” is the sequence of nucleic acid mRNA that is translated into protein. It is referred to as open to contrast reading frames containing termination codons.

“Untranslated region” is abbreviated as UTR refers to sections of mRNA flanking the ORF. On the 5′-side it is the 5′-UTR or leader and on the 3′-side it is the 3′-UTR or trailer. The 5′-UTR contains sequence that is recognized by the ribosome and facilitates the initiation of translation.

The 3′-UTR is located immediately after the translation termination codon and facilitates post-transcriptional modification.

“Kozak consensus sequence” is this sequence that contains the initiation start of translation and contains the sequence 5′-ACCAUGG-3′ where the AUG is the first methionine codon in an mRNA transcript.

The sections herein will describe methods of administering the vaccine, methods of producing the vaccines, compositions comprising the vaccines, and nucleic acids encoding the vaccines. In some embodiments the vaccine may be compromised of an adjuvant as describe herein.

Individuals with coronary artery disease experience inflammatory, apoptotic, and proliferative processes associated with the expression of a soluble form of the tumor necrosis factor-alpha receptor II (sTNFR2). The sTNRF2 is distinct in pathological responses from the membrane-bound mTNFR2 (the “wild type” form). The soluble sTNFR2 is synthesized as a splice variant from TNFR2 pre-mRNA in which the membrane-binding domain of the protein associated with exon 7 (a cassette exon) is excluded from the mature mRNA resulting in a truncated protein with no membrane-binding domain. Alternately, sTNFR2 can be created by peptidase cleavage excluding amino acids associated with exons 7 and 8 from TNFR2 protein leading to a truncated protein with no membrane-binding domain. The unique joining of exon 6 to exon 8 (Δ7sTNFR2) and/or the unique joining of exon 6 to exon 9 (Δ7,8 TNFR2) creates a novel vaccine antigen that will only recognize Δ7sTNFR2 and/or Δ7,8 TNFR2 but not recognizing the “wild-type” TNFR2 (mTNFR2) expression. The vaccine antigen is limited to 14 amino acids flanking the novel exon 6 to 8 junction and/or the exon 6 to 9 junction. In this way, only the pathogenic forms of the sTNFR2 will be acted upon resulting in their clearance from the body. This precision vaccine limits adverse events by evading the other forms of TNFα and TFNR2. The composition of the vaccine is described as an mRNA vaccine as a preferred embodiment, but a peptide vaccine is also described. The vaccine's intended use is for treating patients with vulnerable and/or stable atherosclerotic plaque to prevent progression to major adverse cardiac events (MACE). Multiple delivery options and formulations are also described.

Described herein is the composition and administration of a therapeutic vaccine systemically or locally to an atherosclerotic plaque via a drug-eluting stent, local administration by local catheter delivery systems, intra-coronary intraluminal delivery via standard or specially delivery systems and systemic delivery or other suitable means in patients with atherosclerotic disease in different locations and different stages, stable and unstable forms.

One embodiment described herein utilizes the unique joining of exon 6 to exon 8 (Δ7sTNFR2) and/or the unique joining of exon 6 to exon 9 (Δ7,8 TNFR2) to create an antigen in a vaccine that will only recognize Δ7sTNFR2 and/or Δ7,8 TNFR2 and not recognizing the “wild-type” TNFR2 expression. In this way, only the pathogenic forms of the sTNFR2 will be acted upon, resulting in their clearance from the body. The precision vaccine is designed to limit adverse events by evading other forms of TNFα and TFNR2.

### Current Therapies

Inflammation, atherothrombosis, and coronary syndromes are interdependent. Anti-inflammatory drugs are used in the treatment of ischemic heart disease, atherosclerosis, and unstable plaque. Aspirin reduces the inflammation and platelet aggregation and risk of major cardiovascular events. Recent clinical trials find the inhibition of interleukin 1β (IL-1β) with subcutaneous canakinumab and NLRP3 inflammasome inhibition with colchicine can present major adverse cardiac events (MACE). Andreotti et al., Eur. Heart J. Suppl. 23(Suppl E): E13-E8 (2021). Administration of these anti-inflammatory agents is associated with reductions in key biomarkers including C-reactive protein, fibrinogen, plasminogen activator inhibitor type I, and von Willebrand factor.

Anti-inflammatory therapy involves chronic treatment and may be interrupted by lack of compliance, cost considerations, and occasional adverse reactions. Vaccination against atherosclerosis may replace chronic drug therapy and has been an area of investigation for the last few decades. Amirfakhryan, Hellenic J. Cardiol. 61(2): 78-91 (2020). Vaccine antigens have focused on lipids such as Ox-LDL, ApoB100, and PKSK9 as well as non-lipid antigens including HSPs, β2GPI, and interleukins. Critical in the search for an effective vaccine is the identification of antigens that are not associated with maintaining physiological homeostasis. Antigens epitopes that are variants of wild-type proteins only expressed in pathological conditions associated with coronary artery disease are most likely to succeed.

Another embodiment described herein is an RNA vaccine comprising a nucleotide sequence selected from one or more of SEQ ID NO: 5, 7, 9, 11, or 13-16 and one or more pharmaceutically acceptable excipients. In one aspect, the nucleotide sequence is selected from SEQ ID NO: 15 or 16. In another aspect, the nucleotide sequence comprises one or more modifications to the ribose sugar, the nucleotide base, the 5′-terminus, the 3′-terminus, or combinations thereof. In another aspect, the vaccine is a nanoparticle formulation.

Another embodiment described herein is a peptide vaccine comprising a polypeptide sequence selected from one or more of SEQ ID NO: 6, 8, 10, or 12 and one or more pharmaceutically acceptable excipients. In one aspect, the polypeptide sequence is SEQ ID NO: 6. In another aspect, the polypeptide sequence comprises one or more modifications to the peptide backbone, the amino acid side chains, the N-terminus, the C-terminus, or combinations thereof. In another aspect, the vaccine is a nanoparticle formulation.

Another embodiment described herein is a vascular stent, comprising an RNA vaccine comprising a nucleotide sequence selected from one or more of SEQ ID NO: 5, 7, 9, 11, or 13-16; or eptide vaccine comprising a polypeptide sequence selected from one or more of SEQ ID NO: 6, 8, 10, or 12; and one or more pharmaceutically acceptable excipients. In one aspect, the vaccine is immediately released or controlled released.

Another embodiment described herein is leaflets of a heart valve, comprising an RNA vaccine comprising a nucleotide sequence selected from one or more of SEQ ID NO: 5, 7, 9, 11, or 13-16; or eptide vaccine comprising a polypeptide sequence selected from one or more of SEQ ID NO: 6, 8, 10, or 12; and one or more pharmaceutically acceptable excipients. In one aspect, the vaccine is immediately released or controlled released.

Another embodiment described herein is a drug eluting balloon, comprising an RNA vaccine comprising a nucleotide sequence selected from one or more of SEQ ID NO: 5, 7, 9, 11, or 13-16; or eptide vaccine comprising a polypeptide sequence selected from one or more of SEQ ID NO: 6, 8, 10, or 12; and one or more pharmaceutically acceptable excipients. In one aspect, the vaccine is immediately released or controlled released.

Another embodiment described herein is a method for treating atherothrombosis, the method comprising, administering a therapeutically effective amount of an RNA vaccine comprising a nucleotide sequence selected from one or more of SEQ ID NO: 5, 7, 9, 11, or 13-16; or eptide vaccine comprising a polypeptide sequence selected from one or more of SEQ ID NO: 6, 8, 10, or 12; and one or more pharmaceutically acceptable excipients, or implanting a vascular stent, a heart valve, a drug eluting balloon comprising an RNA vaccine comprising a nucleotide sequence selected from one or more of SEQ ID NO: 5, 7, 9, 11, or 13-16; or eptide vaccine comprising a polypeptide sequence selected from one or more of SEQ ID NO: 6, 8, 10, or 12; and one or more pharmaceutically acceptable excipients to a subject in need thereof. In one aspect, the vaccine is administered four times over a period of 16 weeks. In another aspect, the vaccine is administered at the initial administration, at 4 weeks, at 10 weeks, and at 16 weeks. In another aspect, the vaccine dose is 30 μg to 100 μg mg.

Another embodiment described herein is a method for treating atherothrombosis, the method comprising, administering a therapeutically effective amount of a vaccine an RNA vaccine comprising a nucleotide sequence selected from one or more of SEQ ID NO: 5, 7, 9, 11, or 13-16; or eptide vaccine comprising a polypeptide sequence selected from one or more of SEQ ID NO: 6, 8, 10, or 12; and one or more pharmaceutically acceptable excipients, locally directly to atherosclerotic plagues using micro catheters.

It will be apparent to one of ordinary skill in the relevant art that suitable modifications and adaptations to the compositions, formulations, methods, processes, and applications described herein can be made without departing from the scope of any embodiments or aspects thereof. The compositions and methods provided are exemplary and are not intended to limit the scope of any of the specified embodiments. All of the various embodiments, aspects, and options disclosed herein can be combined in any variations or iterations. The scope of the compositions, formulations, methods, and processes described herein include all actual or potential combinations of embodiments, aspects, options, examples, and preferences herein described. The exemplary compositions and formulations described herein may omit any component, substitute any component disclosed herein, or include any component disclosed elsewhere herein. The ratios of the mass of any component of any of the compositions or formulations disclosed herein to the mass of any other component in the formulation or to the total mass of the other components in the formulation are hereby disclosed as if they were expressly disclosed. Should the meaning of any terms in any of the patents or publications incorporated by reference conflict with the meaning of the terms used in this disclosure, the meanings of the terms or phrases in this disclosure are controlling. Furthermore, the foregoing discussion discloses and describes merely exemplary embodiments. All patents and publications cited herein are incorporated by reference herein for the specific teachings thereof.

Various embodiments and aspects of the inventions described herein are summarized by the following clauses:
Clause 1. An RNA vaccine comprising a nucleotide sequence selected from one or more of SEQ ID NO: 5, 7, 9, 11, or 13-16 and one or more pharmaceutically acceptable excipients.
Clause 2. The vaccine of clause 1, wherein the nucleotide sequence is selected from SEQ ID NO: 15 or 16.
Clause 3. The vaccine of clause 1 or 2, wherein the nucleotide sequence comprises one or more modifications to the ribose sugar, the nucleotide base, the 5′-terminus, the 3′-terminus, or combinations thereof.
Clause 4. The vaccine of any one of clauses 1-3 wherein the vaccine is a nanoparticle formulation.
Clause 5. A peptide vaccine comprising a polypeptide sequence selected from one or more of SEQ ID NO: 6, 8, 10, or 12 and one or more pharmaceutically acceptable excipients.
Clause 6. The vaccine of clause 5, wherein the polypeptide sequence is SEQ ID NO: 6.
Clause 7. The vaccine of clause 5 or 6, wherein the polypeptide sequence comprises one or more modifications to the peptide backbone, the amino acid side chains, the N-terminus, the C-terminus, or combinations thereof.
Clause 8. The vaccine of any one of clauses 5-7, wherein the vaccine is a nanoparticle formulation.
Clause 9 A vascular stent, comprising the vaccine of any one of clauses 1-8.

Clause 10. The vascular stent of clause 9, wherein the vaccine is immediately released or controlled released.

Clause 11. Leaflets of a heart valve, comprising the vaccine of any one of clauses 1-8

Clause 12. The leaflets of a heart valve of clause 11, wherein the vaccine is immediately released or controlled released.

Clause 13. A drug eluting balloon, comprising the vaccine of any one of clauses 1-8.

Clause 14. The drug eluting balloon of clause 13, wherein the vaccine is immediately released or controlled released.

Clause 15. A method for treating atherothrombosis, the method comprising, administering a therapeutically effective amount of a vaccine of any one of clauses 1-8, the vascular stent of clause 9 or 10, the leaflets of the heart valve of clause 11 or 12, the drug eluting balloon of clause 13 or 14 to a subject in need thereof.

Clause 16. The method of clause 15, wherein the vaccine is administered four times over a period of 16 weeks.

Clause 17. The method of clause 15, wherein the vaccine is administered at the initial administration, at 4 weeks, at 10 weeks, and at 16 weeks.

Clause 18. The method of clause 15, wherein the vaccine dose is 30 μg to 100 μg mg.

Clause 19. A method for treating atherothrombosis, the method comprising, administering a therapeutically effective amount of a vaccine of any one of clauses 1-7, locally directly to atherosclerotic plagues using micro catheters.

### EXAMPLES

### Design, Synthesis, and Production

### Pharmaceutical Vaccine Compositions

The amino acids indicated contain a single epitope for immune response that will not overlap with any wild-type protein epitopes to ensure selectivity for the indicated TNFα variant.

TABLE 1
Antigen mRNA and Encoded Peptide Sequences

| | | | SEQ ID |
|---|---|---|---|
| Name | mRNA Sequence (5′→3′) | Peptide Sequence | NO^{†} |
| Δ7TNFR2 | GAC UUC GCC CUG CCC GUG G/AG | DFALPVEKPLCLOR | 1, 2 |
| | AAG CCC CUG UGC CUG CAG AGA | | |
| Δ7,8TNFR2 | ACC GGC GAC UUC GCC CUG CCC | TGDFALPVASLACR | 3, 4 |
| | GUG/GCC AGU CUG GCC UGC AGA | | |

The ″/″ indicates an exon splice site and underlined nucleotides are those improved by codon optimization.
^{†}The odd sequence is for the mRNA sequence and the even sequence is for the polypeptide encoded by the mRNA.

### Pharmaceutical Formulations

### Intravenous Injections

Phosphate buffered saline (PBS) containing inactive ingredients-potassium phosphate mono basic, anhydrous, USP; potassium chloride, USP; sodium phosphate diabetic, anhydrous, USP; sodium chloride, USP; and water for injection, USP. Active ingredients include mRNA with and without antisense IL-10E4SA. The phosphate buffered saline with and without enhanced delivery agents including lipid nanoparticles (LNP) including but not limited to lipids (including ((4-hydroxybutyl)azanediyl)bis (hexane-6,1-diyl)bis (2-hexyldecanoate), 2[(polyethylene glycol)-2000]-N,N-ditetradecylacetamide, 1,2-distearoyl-sn-glycero-3-phosphocholine, and cholesterol), perflourocarbon micro bubbles (C4F10 or C5F12), or cationic peptides including but not limited to ArgArgArgArgArgArgGly (R₆G) (SEQ ID NO: 17).

### Intramuscular Injections

A PBS solution as described for intravenous injection including mRNA with and without antisense IL-10E4SA administered in a solution of less than one milliliter into a muscle, e.g., the shoulder. The PBS solution with or without enhanced delivery agents including lipid nanoparticles (LNP) or cationic peptides including but not limited to R₆G.

### Inhalation

A PBS solution as described for intravenous injection including mRNA with and without antisense IL-10E4SA administered by an aerosol spray delivery device intra-nasally.

A dry powder composed of aerodynamic particle size distribution—a fine particle fraction (FPF) greater than 50 percent, a mean mass aerodynamic diameter (MMAD) of 2.0-2.5 micrometers, emitted dose (ED) of greater than 35% neat mRNA with and without IL-10E4SA and salt. The dry powder will be delivered by a flow-controlled inhalation metered device.

### Oral

An oral rehydration solution (ORT) composed of 2.6 g NaCl, 2.9 g trisodium citrate, 1.5 g potassium chloride, 13.5 g anhydrous glucose, and mRNA with and without antisense IL-10E4SA in one liter of water.

An oral solid dosage (pills or capsules) containing mRNA with or without antisense IL-10E4SA and excipients including binders, glidants, disintegrants, and lubricants to facilitate fill formation and dissolution in the gut.

### Formulations, Administration, Delivery, and Dosing

### Formulations

The linear mRNA can be formulated by multiple strategies indicated in this document. Patients seeking vaccination will have confirmed disease and will be screened for prior adverse reactions to vaccines to exclude patients that may experience serious adverse reactions. No concurrent chemotherapy is permitted due to likelihood of immune suppressive actions of chemotherapy that may limit the efficacy of an mRNA vaccine. Both male and female patients of all ethnic and ideological groups are included.

### Administration

The Δ7TNFR2 mRNA vaccine is administered by routes of administration such as intramuscular at doses of 0.03 to 0.10 mg of the ΔTNFR2 mRNA vaccine. The initial vaccination is designated day 0 and additional vaccinations will be administered at week 4, week 10, and week 16 to boost vaccine response. Patients are monitored for disease progression by standard procedures under the care of a qualified cardiologist.

TABLE 2
Exemplary Administration Protocol

| Day | Day 0 | Week 4 | Week 10 | Week 16 |
|---|---|---|---|---|
| Dose | Initial | Booster+ | Booster+ | Booster+ |

### Delivery Methods

Direct pulmonary delivery (e.g., aerosol, inhalers, etc.) is a more selective mode of drug delivery that typically requires a lower quantity of drug. But it can have limited efficacy due to improper dosing, stability issues, and difficulty in producing an optimum particle size. Pulmonary drug delivery can provide the following advantages: quick onset of action coupled with ease and convenience of administration; the pulmonary dose is significantly lower than the oral dose; and degradation of the drug in the liver can be avoided. On the other hand, the following drawbacks are often associated with pulmonary drug delivery: improper dosing; stability problems; and difficulty in producing the optimum particle size. In addition, not all drugs can be delivered via a pulmonary route due to formulation difficulties.

Local therapeutic administration to atherosclerotic plagues can be buttressed by using different types of drug delivery vehicles (nanoparticle drug carriers, liposomes, viral vectors, or microbubbles). The latter adhere to sites of damaged vascular endothelium and thus may be a method of systemically targeting delivery of therapeutics to organ damaged. For example, perfluorobutane gas microbubbles with a coating of dextrose and albumin efficiently bind to different pharmaceuticals. These 0.3-10.0 pm particles bind to sites of vascular injury. Further, the perfluorobutane gas is an effective cell membrane fluidizer. The potential advantages of microbubble carrier delivery include none to minimal (additional) vessel injury through delivery, no resident polymer to degrade and lead to eventual inflammation, rapid bolus delivery, and repeated delivery. Microbubble carriers were successfully used in different animal models and clinical trials to deliver antisense oligonucleotide and/or Sirolimus to the injured vascular bed.

### Extracellular Vesicles (EV)

Kumar and colleagues describe the use of extracellular vesicles (EVs), which are a family of natural carriers in the human body. EVs play a critical role in cell-to-cell communications and can be used as unique drug carriers of therapeutic vaccine to tumors. Though the authors of the reported investigations concluded that certain limitations need to be overcome as well as understanding the mechanism to control targeted delivery. Specifically, the isolation and drug encapsulation techniques employed to engineer EVs could result in the loss of functional properties of the EVs, such as the destruction of surface proteins. These unintended changes could lead to nonspecific interactions with other cells, leading to off-target effects, toxicity, and suboptimal efficacy.

### Adenosine Nanoparticles

Recently, the efficacy in mitigating inflammation was demonstrated through the targeted delivery of adenosine and of multi-drug formulations. Bioconjugation of adenosine to squalene produces a prodrug-based nanocarrier, which, after nano formulation with α-tocopherol (vitamin E), yields stable multidrug nanoparticles. This nanoparticle improves the bioavailability of both drugs with significant pharmaceutical activity in models of acute inflammatory injury.

### Novel Bio-Objects

A group of researchers has succeeded in engineering a new kind of microscopic bio-object that may one day be used for personalized diagnostics and targeted delivery of drugs. The object consists of a genetically modified E. coli bacterium and nano-erythrocytes (small vesicles made of red blood cells), and it demonstrates a substantial improvement in motility over previous designs.

### Nanobodies

In some embodiments therapeutic vaccine can be delivered using nanobodies. Indeed, several researchers have shown that nanobodies—which are tiny immune proteins can enhance site specific delivery and residence of vaccines.

### Nanomicells

In brain tumors vaccine maybe delivered brain-derived neurotrophic factor mRNA using polyplex nanomicelle.

Ischemic neuronal death causes serious lifelong neurological deficits; however, there is no proven effective treatment that can prevent neuronal death after the ischemia. We investigated the feasibility of mRNA therapeutics for preventing the neuronal death in a rat model of transient global ischemia (TGI). By intraventricular administration of mRNA encoding brain-derived neurotrophic factor (BDNF) using a polymer-based carrier, polyplex nanomicelle, the mRNA significantly increased the survival rate of hippocampal neurons after TGI, with a rapid rise of BDNF in the hippocampus.

The nanomicelle has a core-shell structure surrounded by a PEG outer shell and an mRNA-containing core for stable retention of the mRNA in the nanomicelle. The local administration of mRNA loaded nanomicelles has already shown therapeutic potential in various organs, such as the liver, joint cartilage, intervertebral disk, and the neural tissues, including the brain nanomicelles can also block the immune responses to extracellular mRNA by shielding them from recognition by the toll-like receptors in target cells.

In some embodiments the problems associated with systemic delivery may be overcome by using intra-arterial or intravenous selective delivery of therapeutic agents using a percutaneous trans-catheter route to deliver a therapeutic agent for treating solid organ tumors.

In some embodiments, a micro-catheter is introduced into a damaged artery and/or its branches and a therapeutic agent for treating is infused through the micro-catheter. In some embodiments, the therapeutic agent is infused via an intravenous and/or intraarterial route using a micro-catheter.

Different configurations of micro-catheters may be used. Any of the pharmaceuticals mentioned above may be delivered in a liquid state via the subject's blood, optionally with mixture of contrast agent and/or saline.

Alternatively, A drug-eluting stent (DES) may be used. The stent is coated with a special polymer that contains medication. The polymer coating helps control the release of the drug into the surrounding tissue.

In some embodiments a drug-eluting balloon (DEB) may be used in the treatment of coronary artery disease and peripheral artery disease. This comprises a balloon catheter that combines the properties of a traditional balloon angioplasty with the delivery of medication to the affected artery. A drug-eluting balloon consists of a catheter with an inflatable balloon. The balloon is coated with a medication. This medication is embedded in a special matrix or coating on the surface of the balloon. As the balloon is inflated, the medication is released and comes into contact with the arterial wall. The drug is transferred to the vessel wall and exerts its pharmacological effects locally.

The potential benefits of drug-eluting Transcatheter Aortic Valve Replacement (TAVR) valves would be similar to those of drug-eluting stents or drug-eluting balloons, which involve the local delivery of medication to improve the long-term performance and durability of the implanted device.

The use of drug-eluting technology in TAVR valves could help address complications such as valve degeneration, calcification, or inflammation, which may impact the durability and function of the prosthetic valve over time. By delivering medication directly to the valve, it may be possible to prevent or delay these complications, potentially improving valve longevity and reducing the need for repeat interventions.

Alternatively, different carriers such as micro-particles, nanoparticles, injectable polymers or natural carriers and others may be used to enhance penetration and residence of therapeutics at the target area.

To avoid the disadvantages of oral or direct injection administration of drugs, a number of modes of administration of continuous dose, long-term delivery devices include reservoir devices, osmotic devices, and pulsatile devices for delivering beneficial agents have been utilized. Injecting drug delivery systems as small particles, microparticles or microcapsules avoids the incision needed to implant drug delivery systems. Microparticles, microspheres, and microcapsules, referred to herein collectively as “microparticles,” are solid or semi-solid particles having a diameter of less than one millimeter, more preferably less than 100 μm, which can be formed of a variety of materials, including synthetic polymers, and proteins. Another intensively studied polymeric injectable depot system is an in-situ-forming implant system. In situ-forming implant systems are made of biodegradable products, which can be injected via a syringe into the body, and once injected, congeal to form a solid biodegradable implant.

### Dosing

The dosing of the heart vaccine may range from 0.03 mg to 0.10 mg.

TABLE 3
Sequences

| | | SEQ ID |
|---|---|---|
| Description | Sequence (5′→3′ or N→C) | NO: |
| Soluble Tumor Necrosis Factor | GACUUCGCUCUUCCAGUUG/ | 5 |
| Receptor 2 Exons 6/8 Δ7 | AGAAGCCCUUGUGCCUGCAGAGA | |
| mRNA; Sequence Length: 42 | | |
| Soluble Tumor Necrosis Factor | DFALPVEKPLCLOR | 6 |
| Receptor 2 Δ7 sTNFR2; | | |
| Peptide Sequence Length: 14 | | |
| Soluble Tumor Necrosis Factor | ACUGGCGACUUCGCUCUUCCAGUU/ | 7 |
| Receptor 2 Exons 6/9 Δ7,8 | GCCUCACUUGCCUGCCGA | |
| mRNA; Sequence Length: 42 | | |
| Soluble Tumor Necrosis Factor | TGDFALPVASLACR | 8 |
| Receptor 2 Δ7,8 DS-sTNFR2; | | |
| Peptide Sequence Length: 14 | | |
| Codon Optimized Δ7TNFR2; | GACUUCGCCCUGCCCGUGG/ | 9 |
| Sequence Length: 42 | AGAAGCCCCUGUGCCUGCAGAGA | |
| Codon Optimized Δ7TNFR2; | DFALPVEKPLCLOR | 10 |
| Peptide Sequence Length: 14 | | |
| Codon Optimized Δ7,8TNFR2; | ACCGGCGACUUCGCCCUGCCCGUG/ | 11 |
| Sequence Length: 42 | GCCAGUCUGGCCUGCAGA | |
| Codon Optimized Δ7,8TNFR2; | TGDFALPVASLACR | 12 |
| Peptide Sequence Length: 14 | | |
| 5′-UTR based on HCV IRES; | GCCAGCCCCCUGAUGGGGGCGACACUCCACCAUGAAUCAC | 13 |
| Sequence Length: 327 | UCCCCCGGCGGGUCUACGUCUUCACGCAGAAAGCGCCUAG | |
| | CCAUGGCGCUAGUAUGAGUGUCGUGCAGCCGCCGGGACCC | |
| | CCCCUCCCGGGAGAGCCAUAGCGGUCUGCGGAACCGGUGC | |
| | ACCGGAAUGCCAGGACGACCGGGUCCUUUCUUGGAUCAAC | |
| | CCGCUCAAUGCCUGGAGAUUUGGGCGUGCCCCCGCAGACC | |
| | GCAAGCCGUAGUGUUGGGUCGCGAAGGCCUUGUGGUACUG | |
| | CCUGAUAGGGUGCUUGCGAGUGCCCCUUUAGGUCUCGUAG | |
| | ACCGUGC | |
| 3′-UTR based on beta-globin; | GCUCGCUUUCUUGCUGUCCAAUUUCUAUUAAAGGUCCUUU | 14 |
| Sequence Length: 131 | GUUCCCUAAGUCCAACUACUAAACUGAGGGAUAUUACAAA | |
| | GGGCCUUGAGCAUCUGGAUUCUGCCUAAUAAAAAACAUUU | |
| | AUUUUCAUUGC | |
| Δ7 sTNFR2 (5′-UTR, ORF, 3′- | GCCAGCCCCCUGAUGGGGGCGACACUCCACCAUGAAUCAC | 15 |
| UTR); Sequence Length: 506 | UCCCCCGGCGGGUCUACGUCUUCACGCAGAAAGCGCCUAG | |
| | CCAUGGCGCUAGUAUGAGUGUCGUGCAGCCGCCGGGACCC | |
| | CCCCUCCCGGGAGAGCCAUAGCGGUCUGCGGAACCGGUGC | |
| | ACCGGAAUGCCAGGACGACCGGGUCCUUUCUUGGAUCAAC | |
| | CCGCUCAAUGCCUGGAGAUUUGGGCGUGCCCCCGCAGACC | |
| | GCAAGCCGUAGUGUUGGGUCGCGAAGGCCUUGUGGUACUG | |
| | CCUGAUAGGGUGCUUGCGAGUGCCCCUUUAGGUCUCGUAG | |
| | ACCGUGCAUGGACUUCGCCCUGCCCGUGGAGAAGCCCCUG | |
| | UGCCUGCAGAGAUAAGCUCGCUUUCTTGCTGTCCAATTTC | |
| | TATTAAAGGTCCTTTGTTCCCTAAGTCCAACTACTAAACT | |
| | GAGGGATATTACAAAGGGCCTTGAGCATCTGGATTCTGCC | |
| | TAATAAAAAACATTTATTTTCATTGC | |
| Δ7,8 DS sTNFR2 (5′-UTR, | GCCAGCCCCCUGAUGGGGGCGACACUCCACCAUGAAUCAC | 16 |
| ORF, 3′-UTR); Sequence | UCCCCCGGCGGGUCUACGUCUUCACGCAGAAAGCGCCUAG | |
| Length: 506 | CCAUGGCGCUAGUAUGAGUGUCGUGCAGCCGCCGGGACCC | |
| | CCCCUCCCGGGAGAGCCAUAGCGGUCUGCGGAACCGGUGC | |
| | ACCGGAAUGCCAGGACGACCGGGUCCUUUCUUGGAUCAAC | |
| | CCGCUCAAUGCCUGGAGAUUUGGGCGUGCCCCCGCAGACC | |
| | GCAAGCCGUAGUGUUGGGUCGCGAAGGCCUUGUGGUACUG | |
| | CCUGAUAGGGUGCUUGCGAGUGCCCCUUUAGGUCUCGUAG | |
| | ACCGUGCAUGACCGGCGACUUCGCCCUGCCCGUGGCCAGU | |
| | CUGGCCUGCAGAUAAGCUCGCUUUCTTGCTGTCCAATTTC | |
| | TATTAAAGGTCCTTTGTTCCCTAAGTCCAACTACTAAACT | |
| | GAGGGATATTACAAAGGGCCTTGAGCATCTGGATTCTGCC | |
| | TAATAAAAAACATTTATTTTCATTGC | |

Each of the sequences shown above can be modified. The nucleotide sequences can comprise one or more modifications known in the art to the ribose sugar, the nucleotide base, the 5′-terminus, the 3′-terminus, or combinations thereof. The polypeptide sequences can comprise one or more modifications know in the art to peptide backbone, the amino acid side chains, the N-terminus, the C-terminus, or combinations thereof.

### Modifications

RNA synthesized to include 2′-O-methylated ribose, 5-methyl-uridines, or pseudouridine nucleosides do not activate the oligoadenylate synthase (OAS) sensing pathway and induction of the RNase L nuclease resulting in more durable translation of the mRNA vaccine.

Synthetic mRNA with a 5′-end modification, a 5′-CAP, confers greater mRNA vaccine stability and enhanced translatability. The natural cap structure is an N⁷ -methylated guanosine (m⁷G) connected by a 5′-to 5′-O-triphospohate bridge. In some cases, a 2′-O-methylation of the +1 nucleotide ribose results in a cap structure m⁷GpppN²′OmeN. The synthesis of the natural cap can be complex and an alternate synthetic “CleanCap AG trimer” (Tri Link Biotechologies, cat no. N-7113) can be added in vitro using a T7 RNA polymerase. Henderson et al. Curr. Protoc. 1(2): e39 (2021).

What is claimed is:

1. An RNA vaccine comprising a nucleotide sequence selected from one or more of SEQ ID NO: 5, 7, 9, 11, or 13-16 and one or more pharmaceutically acceptable excipients.

2. The vaccine of claim 1, wherein the nucleotide sequence is selected from SEQ ID NO: 15 or 16.

3. The vaccine of claim 1, wherein the nucleotide sequence comprises one or more modifications to the ribose sugar, the nucleotide base, the 5′-terminus, the 3′-terminus, or combinations thereof.

4. The vaccine of of claim 1 wherein the vaccine is a nanoparticle formulation.

5. A peptide vaccine comprising a polypeptide sequence selected from one or more of SEQ ID NO: 6, 8, 10, or 12 and one or more pharmaceutically acceptable excipients.

6. The vaccine of claim 5, wherein the polypeptide sequence is SEQ ID NO: 6.

7. The vaccine of claim 6, wherein the polypeptide sequence comprises one or more modifications to the peptide backbone, the amino acid side chains, the N-terminus, the C-terminus, or combinations thereof.

8. The vaccine of claim 5, wherein the vaccine is a nanoparticle formulation.

9. A vascular stent, comprising an RNA vaccine comprising a nucleotide sequence selected from one or more of SEQ ID NO: 5, 7, 9, 11, or 13-16; or eptide vaccine comprising a polypeptide sequence selected from one or more of SEQ ID NO: 6, 8, 10, or 12; and one or more pharmaceutically acceptable excipients.

10. The vascular stent of claim 9, wherein the vaccine is immediately released or controlled released.

11. Leaflets of a heart valve, comprising an RNA vaccine comprising a nucleotide sequence selected from one or more of SEQ ID NO: 5, 7, 9, 11, or 13-16; or eptide vaccine comprising a polypeptide sequence selected from one or more of SEQ ID NO: 6, 8, 10, or 12; and one or more pharmaceutically acceptable excipients.

12. The leaflets of a heart valve of claim 11, wherein the vaccine is immediately released or controlled released.

13. A drug eluting balloon, comprising an RNA vaccine comprising a nucleotide sequence selected from one or more of SEQ ID NO: 5, 7, 9, 11, or 13-16; or eptide vaccine comprising a polypeptide sequence selected from one or more of SEQ ID NO: 6, 8, 10, or 12; and one or more pharmaceutically acceptable excipients.

14. The drug eluting balloon of claim 13, wherein the vaccine is immediately released or controlled released.

15. A method for treating atherothrombosis, the method comprising, administering a therapeutically effective amount of an RNA vaccine comprising a nucleotide sequence selected from one or more of SEQ ID NO: 5, 7, 9, 11, or 13-16; or eptide vaccine comprising a polypeptide sequence selected from one or more of SEQ ID NO: 6, 8, 10, or 12; and one or more pharmaceutically acceptable excipients, or implanting a vascular stent, a heart valve, a drug eluting balloon comprising an RNA vaccine comprising a nucleotide sequence selected from one or more of SEQ ID NO: 5, 7, 9, 11, or 13-16; or eptide vaccine comprising a polypeptide sequence selected from one or more of SEQ ID NO: 6, 8, 10, or 12; and one or more pharmaceutically acceptable excipients to a subject in need thereof.

16. The method of claim 15, wherein the vaccine is administered four times over a period of 16 weeks.

17. The method of claim 15, wherein the vaccine is administered at the initial administration, at 4 weeks, at 10 weeks, and at 16 weeks.

18. The method of claim 15, wherein the vaccine dose is 30 μg to 100 μg mg.

19. A method for treating atherothrombosis, the method comprising, administering a therapeutically effective amount of a vaccine an RNA vaccine comprising a nucleotide sequence selected from one or more of SEQ ID NO: 5, 7, 9, 11, or 13-16; or eptide vaccine comprising a polypeptide sequence selected from one or more of SEQ ID NO: 6, 8, 10, or 12; and one or more pharmaceutically acceptable excipients, locally directly to atherosclerotic plagues using micro catheters.
